(19) United States
(12) Patent Application Publication
(10) Pub. No.: US 2025/0082432 A1
(43) Pub. Date: Mar. 13, 2025

(54) DEVICE FOR CLEANING A CONE OF AN IMPLANT AND SURGICAL SYSTEM

(71) Applicant: Peter Brehm Holding GmbH & Co. KG, Weisendorf (DE)

(72) Inventors: Peter BREHM, Weisendorf (DE); Janick KLING, Aurachtal (DE); Reinhard SCHRODER, Adelsdorf (DE)

(21) Appl. No.: 18/822,349

(22) Filed: Sep. 2, 2024

(30) Foreign Application Priority Data
Sep. 11, 2023 (DE) 202023105231.2

(51) Int. Cl. A61B 90/70; A61F 2/46

(52) U.S. Cl. CPC A61B 90/70; A61F 2/46

(57) ABSTRACT

Device for cleaning a cone of an implant comprises a handle piece, an inner sleeve and an outer sleeve. The handle piece has a through opening extending axially. The through opening has a first section with a larger inner diameter than a second section. The inner sleeve inserts into the second section of the through opening and has a protrusion abutting against an abutment surface between the first and the second sections. The outer sleeve slides over a section of the handle piece. The handle piece and the inner sleeve fix a first part of a textile piece between the protrusion of the inner sleeve and the abutment surface of the handle piece. The handle piece and the outer sleeve fix a second part of the textile piece between the handle piece and the outer sleeve when the outer sleeve is slid over the handle piece.

### FIELD OF THE INVENTION

The present disclosure relates generally to surgical instruments, in particular to devices for cleaning a cone of an implant and to surgical systems comprising such a device.

### BACKGROUND OF THE INVENTION

Endoprostheses are used to replace a patient's natural joint, for example a hip, shoulder or knee joint that has been damaged by degeneration, disease or injury. Endoprostheses are implants that are implanted into the patient's body to replace a joint. To enable better adaptation to the patient's anatomy, there are modular endoprostheses, which are made up of individual modules. The modules are available in different sizes. Depending on the patient's anatomy, suitable modules can be selected and combined with each other. Cone connections can be used to connect the modules, in which a cone on one module is inserted into a cone-shaped recess in another module. The cone and the cone-shaped recess have a small opening angle so that a firm frictional connection is created after inserting the cone into the cone-shaped recess and/or additional tensioning.

Modular hip joint prostheses according to the state of the art, in which conical connections are used, are known, for example, from DE 4 320 086 A1 and EP 2 066 265 A2.

One problem that arises with modular endoprostheses whose modules are connected by cone connections is that the stability of the cone connection can be impaired by contamination between the cone and the cone-shaped recess into which the cone is inserted. Such contamination can occur during the surgery in which the endoprosthesis is implanted in the patient, for example through the patient's blood. The cone must therefore be cleaned before creating the cone connection, e.g. by wiping it with a gauze compress. However, it can happen that impurities are overlooked and not removed.

### SUMMARY OF THE INVENTION OR DISCLOSURE

The present disclosure provides a device with which the cone of an implant can be reliably cleaned of impurities during surgery.

According to a first aspect of the disclosure, a device for cleaning a cone of an implant is provided, which comprises a handle piece, an inner sleeve and an outer sleeve. The handle piece has a proximal end and a distal end, and has a through opening extending axially through the handle piece. The through opening has, at the distal end of the handle piece, a first section which has a larger inner diameter than a second section of the through opening adjoining the first section. The inner sleeve is insertable into the second section of the through opening of the handle piece and has a protrusion at a first end. This is adapted to abut against an abutment surface of the handle piece, which is located at the transition between the first and the second section of the through opening of the handle piece. The outer sleeve is slidable over a distal section of the handle piece at the distal end of the handle piece. The handle piece and the inner sleeve are designed to fix a first part of a textile piece between the protrusion of the inner sleeve and the abutment surface of the handle piece when the inner sleeve is inserted into the second section of the through opening of the handle piece. The handle piece and the outer sleeve are designed to fix a second part of the textile piece between the distal section of the handle piece and the outer sleeve when the outer sleeve is slid over the distal section of the handle piece.

In the device, a textile piece, which can be a gauze compress, for example, can be fixed between the protrusion of the inner sleeve and the abutment surface at the transition between the first and second sections of the through opening of the handle piece, on the one hand, and between the distal section of the handle piece and the outer sleeve, on the other hand, so that it covers the first section of the through opening. To clean the cone of the implant, the device with the textile piece fixed therein can be placed on the cone so that the cone is located in the first section of the through opening of the handle piece. By rotating the device, the textile piece is then moved over the cone of the implant, whereby the cone is cleaned. As the cone is inserted into the first section of the through opening during cleaning, the cone can be cleaned all around, reducing the risk of contaminants remaining on the cone.

According to an embodiment, the device additionally comprises an assembly tool. The assembly tool has, at a first end, a first section which is insertable into the inner sleeve. The first section is adjoined by a second section, which has a larger outer diameter than the first section and is insertable into the first section of the through opening of the handle piece. The second section is adjoined by a third section, which has a larger outer diameter than the second section and is insertable into the outer sleeve.

The assembly tool can be used to assemble the handle piece, the inner sleeve, the outer sleeve and the textile piece. To do this, the outer sleeve is slid over the third section of the assembly tool and the inner sleeve is placed on the first section of the assembly tool. The textile piece is then provided with an opening and the inner sleeve is inserted through the opening of the textile piece. The handle piece is then placed on the assembly tool, wherein the second section of the assembly tool is inserted into the first section of the through opening of the handle piece. In doing so, the textile piece is shaped so that it abuts against the inner wall of the first section of the through opening. When the handle piece is placed on the assembly tool, the textile piece is fixed at the edge of its opening between the protrusion of the inner sleeve and the abutment surface of the handle piece. The outer sleeve is then slid upwards over the distal section of the handle piece, whereby the fixation of the second part of the textile piece occurs between the distal section of the handle piece and the inner wall of the inner sleeve.

According to an embodiment, the assembly tool has a standing surface at a second end for placing the assembly tool on a surface. Thereby, the assembly is facilitated.

According to an embodiment, the device additionally comprises a mandrel with a tip for piercing the textile piece. The mandrel has, at an end facing away from the tip, a holding section which is insertable into the inner sleeve at the second end of the inner sleeve facing away from its first end. The mandrel can be placed on the inner sleeve after the inner sleeve has been placed on the first section of the assembly tool, wherein the holding section of the mandrel is inserted into the inner sleeve. Then, to create the opening of the textile piece, the textile piece is pulled down over the mandrel until it rests on the protrusion of the inner sleeve, and the mandrel is removed again.

According to an embodiment, part of the mandrel has a larger outer diameter than the second section of the through opening of the handle piece, so that the mandrel does not fit through the second section of the through opening. Thereby, the inner sleeve is prevented from being inserted into the second section of the through opening of the handle piece while the mandrel is still on the inner sleeve. Thereby, incorrect assembly of the device can be prevented.

According to an embodiment, the inner sleeve has one or more, in particular three, slots.

According to an embodiment, the inner sleeve has an outwardly curved wall in the region where the slots are arranged, which has an elasticity to hold the inner sleeve inserted into the second section of the through opening of the handle piece to the inner wall of the second section of the through opening of the handle piece. The elastic, outwardly curved wall allows the inner sleeve to be clamped in the second section of the through opening of the handle piece, thereby reducing the risk of the inner sleeve slipping downwardly out of the handle piece. The slots provide a room for movement for the sections of the outwardly curved wall between the slots, allowing them to deform elastically more easily.

According to an embodiment, the distal section of the handle piece has a first region and a second region, each having a larger outer diameter than a third region disposed between the first and second regions. The first and second regions of the handle piece are arranged at the ends of the outer sleeve when the outer sleeve is slid over the distal section of the handle piece. Thereby, the textile piece can be clamped between the first and second regions of the distal section of the handle piece and the inner wall of the sleeve, so that it is fixed at both ends of the outer sleeve. Due to the smaller outer diameter of the third region between the first and second regions of the distal section of the handle piece, there is a space between the third region and the inner wall of the outer sleeve which can capture parts of the textile piece.

According to an embodiment, the distal section of the handle piece in the third region has one or more, in particular two, slots arranged opposite the second end of the inner sleeve and/or the one or more slots of the inner sleeve when the inner sleeve is inserted into the second section of the through opening of the handle piece. Thereby, the inner sleeve can be slid out using a tool inserted through the slots in the distal section of the handle piece in the event that the inner sleeve becomes inadvertently jammed in the handle piece.

According to an embodiment, the second region of the distal section of the handle piece is further away from the distal end of the handle piece than the first region. The outer diameter of the second region of the distal section of the handle piece and an inner diameter of the outer sleeve are configured such that the second region of the distal section of the handle piece forms a limit stop for the outer sleeve when the textile piece is located between the second region of the distal section of the handle piece and the outer sleeve. When the textile piece is not located between the second region of the distal section of the handle piece and the outer sleeve, the outer sleeve is slidable over the second region of the distal section of the handle piece.

The limit stop for the outer sleeve allows the second part of the textile piece to be fixed more securely between the distal section of the handle piece and the outer sleeve. Since the limit stop is only present when the textile piece is located between the second region of the distal section of the handle piece and the outer sleeve, it is noticeable if the textile piece is not properly arranged between the second region of the distal section of the handle piece and the outer sleeve, as the outer sleeve can then be slid further upwards. Thereby, the safety of the device is improved.

According to an embodiment, the handle piece at the proximal end has several axially extending grooves on the outer side of the handle piece. Thereby, holding the handle piece when rotating the device to clean the cone is facilitated.

According to an embodiment, the through opening of the handle piece at the proximal end of the handle piece has an inner diameter that is smaller than an outer diameter of the inner sleeve and smaller than the inner diameter of the second section of the through opening at the distal end of the handle piece. Thereby, the inner sleeve at the proximal end of the handle piece cannot be inserted into the through opening, thereby preventing the handle piece from being placed the wrong way around on the inner sleeve.

According to an embodiment, the first section of the through opening has a corrugation on its wall surface. Thereby, twisting the textile piece during cleaning of the cone can be prevented.

According to an embodiment, the textile piece is a gauze compress. Gauze compresses are suitable for absorbing the patient's blood and other impurities so that impurities can be reliably removed from the cone of the implant.

According to a second aspect of the disclosure, a device for cleaning a cone of an implant is provided, which comprises a handle piece, an inner sleeve, an outer sleeve and a textile piece. The handle piece has a proximal end and a distal end. A through opening extends axially through the handle piece. The through opening has a first section at the distal end of the handle piece, which has a larger inner diameter than a second section of the through opening adjoining the first section. An abutment surface is located at the transition between the first and the second section of the through opening of the handle piece. The inner sleeve is inserted into the second section of the through opening of the handle piece and has a protrusion at a first end. The outer sleeve encloses a distal section of the handle piece at the distal end of the handle piece. The textile piece has an opening through which the inner sleeve is guided. A first part of the textile piece is fixed between the protrusion of the inner sleeve and the abutment surface of the handle piece. In addition, a second part of the textile piece is fixed between the distal section of the handle piece and the outer sleeve.

According to a third aspect of the disclosure, a surgical system comprises a device for cleaning a cone of an implant as described above, and an implant having a cone. The cone of the implant and the first section of the through opening of the handle piece of the device for cleaning a cone of an implant have corresponding shapes.

Since the first section of the through opening of the handle piece and the cone have corresponding shapes, the textile piece is pressed against the entire surface of the cone by the inner wall of the first section of the through opening of the handle piece when cleaning the cone, so that the entire surface of the cone can be reliably cleaned.

According to an embodiment, the implant is an anchoring stem of an endoprosthesis, in particular a hip, shoulder or knee prosthesis.

According to an embodiment, the cone of the implant has an opening with an inner thread. An axis of the opening of the cone of the implant lies on a cone axis of the cone. The system additionally comprises a guide rod, which can be screwed into the opening of the cone of the implant. The guide rod can be guided through the through opening of the handle piece and the inner sleeve of the device inserted into the second section of the through opening of the handle piece for cleaning a cone of an endoprosthesis.

The device is guided by the guide rod while it is placed on the cone of the implant for cleaning. Thereby, placing the device on the cone at an angle is prevented. After cleaning the cone, the guide rod can also be used to place a module with a cone-shaped recess on the cone and to tighten the cone connection.

According to a fourth aspect of the disclosure, a method comprises providing a device for cleaning a cone of an implant. The device comprises: a handle piece with a proximal end, a distal end and a through opening extending axially through the handle piece, wherein the through opening has, at the distal end of the handle piece, a first section which has a larger inner diameter than a second section of the through opening adjoining the first section; an inner sleeve insertable into the second section of the through opening of the handle piece and having at a first end a protrusion for abutment against an abutment surface of the handle piece located at the transition between the first and the second section of the through opening of the handle piece; and an outer sleeve slidable over a distal section of the handle piece at the distal end of the handle piece. The inner sleeve is inserted into an opening in a textile piece, wherein an edge of the opening of the textile piece rests on the protrusion of the inner sleeve. The inner sleeve is inserted into the second section of the through opening of the handle piece, wherein a first part of the textile piece is fixed between the protrusion of the inner sleeve and the abutment surface of the handle piece. The outer sleeve is slid over the distal section of the handle piece, wherein a second part of the textile piece is fixed between the distal section of the handle piece and the outer sleeve.

### BRIEF DESCRIPTION OF THE DRAWINGS

In the following, embodiments of the disclosure are described with reference to the figures.

FIG. 1 shows a schematic cross-sectional view of a device for cleaning a cone of an implant according to an embodiment of the disclosure.

FIG. 2 shows a view of a handle piece of the device for cleaning a cone of an implant.

FIG. 3 shows a schematic view of an inner sleeve of the device for cleaning a cone of an implant.

FIG. 4 shows a schematic view of an outer sleeve of the device for cleaning a cone of an implant.

FIG. 5 shows a schematic view of an assembly tool of the device for cleaning a cone of an implant.

FIG. 6 shows a schematic view of a mandrel of the device for cleaning a cone of an implant.

FIG. 7 shows a schematic view of an implant and a guide rod connected to the implant.

### DETAILED DESCRIPTION OF THE PREFERRED EMBODIMENTS

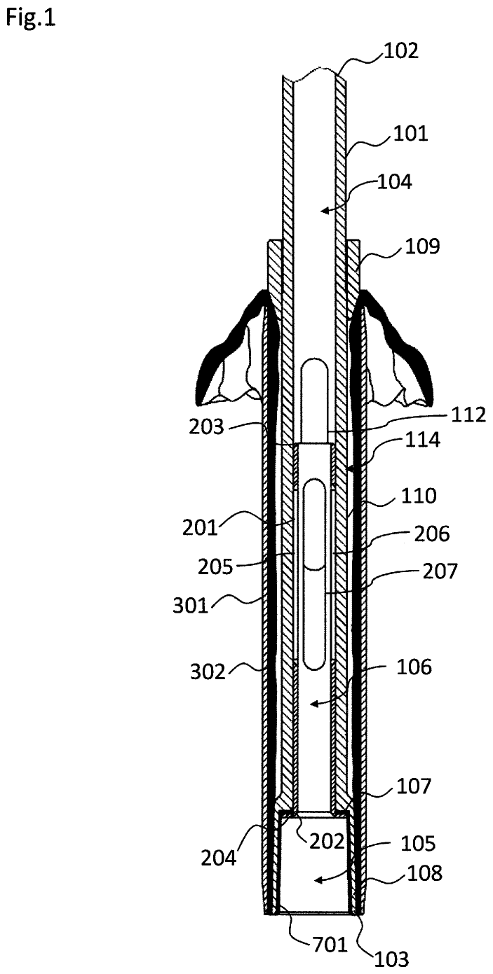

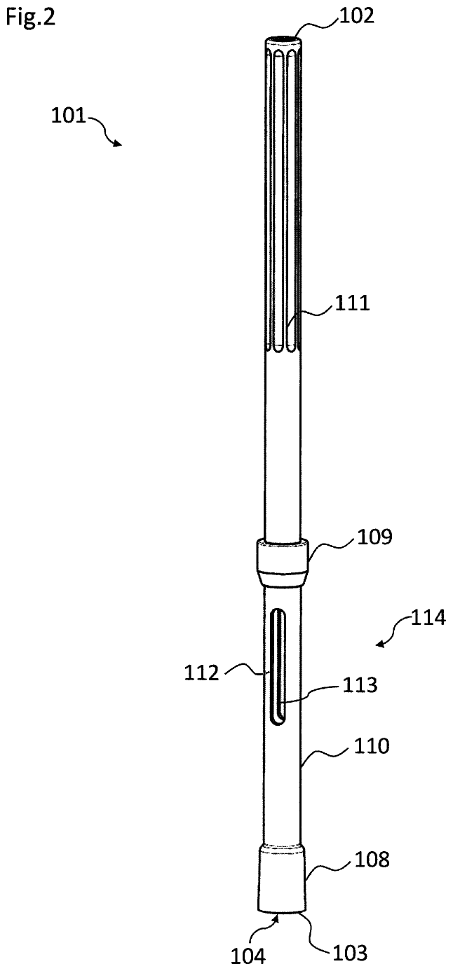

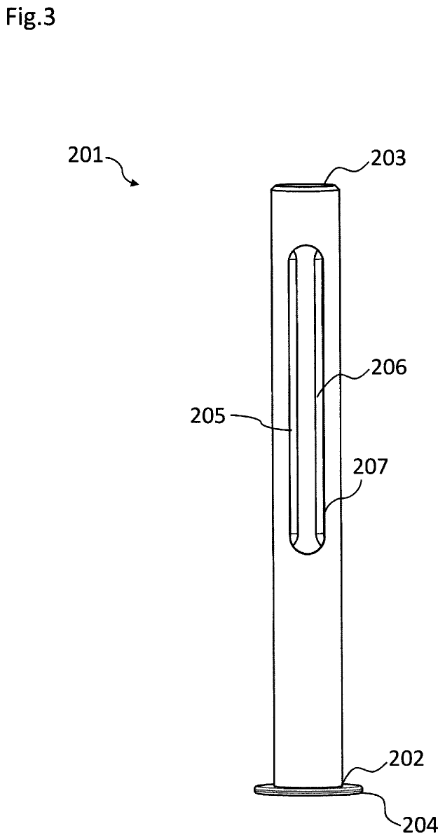

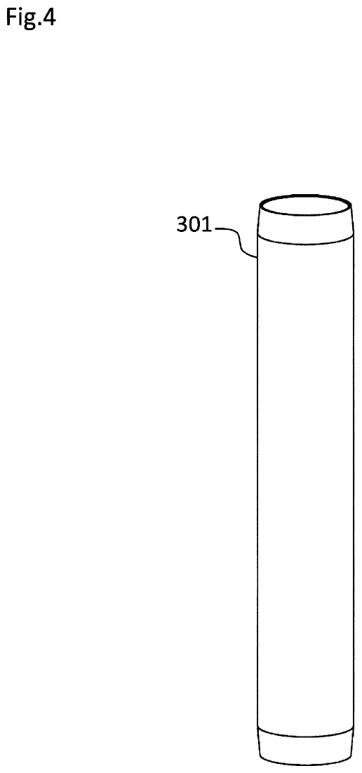

FIG. 1 shows a schematic cross-sectional view of a device for cleaning a cone of an implant according to an embodiment of the disclosure. The device comprises a handle piece 101, an inner sleeve 201 and an outer sleeve 301. Schematic views of the handle piece 101, the inner sleeve 201 and the outer sleeve 301 are shown in FIGS. 2, 3 and 4.

FIG. 1 shows the handle piece 101, the inner sleeve 201 and the outer sleeve 301 in the assembly in which they are located when the device is used to clean the cone of an implant. When the device is used, a textile piece 701 is fixed by the handle piece 101, the inner sleeve 201 and the outer sleeve 301 and is moved over the cone during cleaning of the cone to remove contaminants from the cone. The textile piece 701 can be a gauze compress, for example a 16-fold or 12-fold gauze compress, which when folded has a size of 10×10 cm and is partially unfolded before insertion into the device.

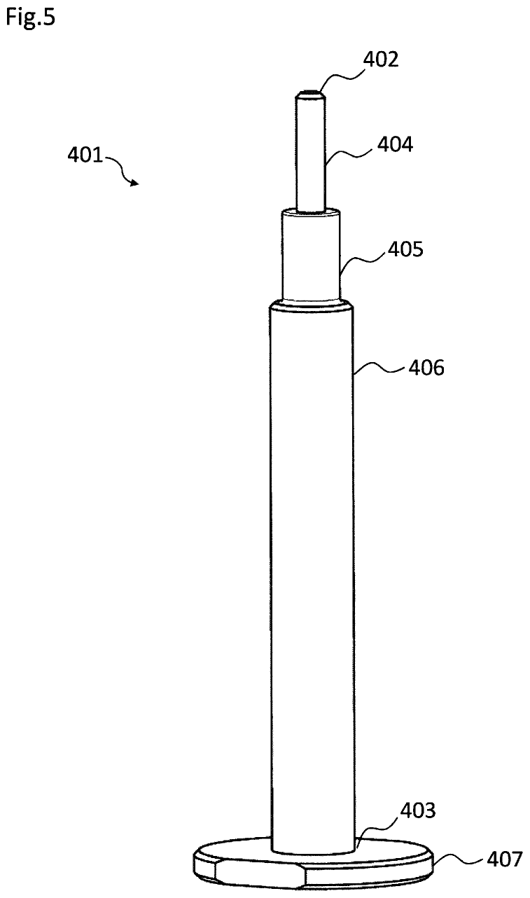

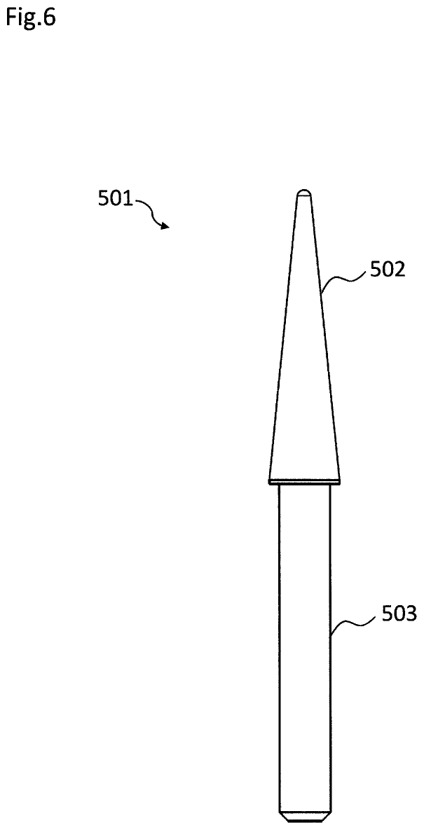

The device according to the embodiment further comprises an assembly tool 401, shown in FIG. 5, and a mandrel 501, shown in FIG. 6.

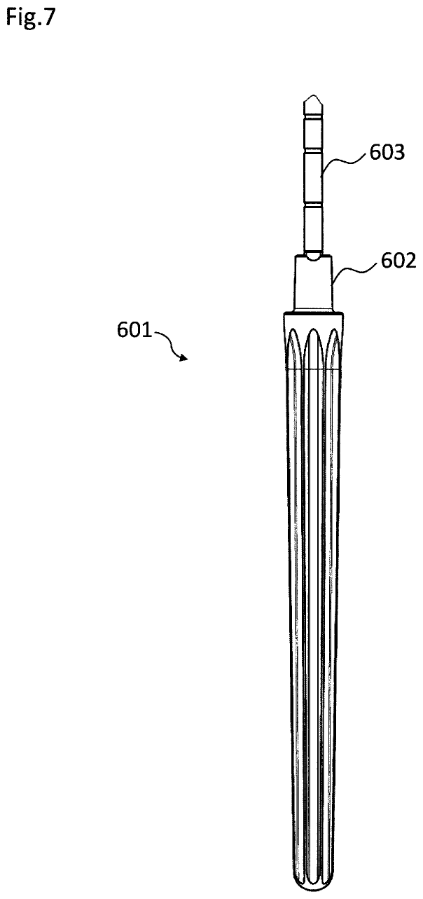

An example of an implant 601 with a cone 602 that can be cleaned with the device according to the embodiment is shown in FIG. 7. The implant 601 shown in FIG. 7 is an anchoring stem. The anchoring stem 601 can be part of an endoprosthesis, for example an artificial hip joint. The anchoring stem 601 is inserted into a long bone, for example the femur, of the patient. A module with a cone-shaped recess corresponding to the shape of the cone 602 can be placed on the cone 602 of the anchoring stem 601, which in turn is connected to a joint ball that is inserted into, for example, an acetabular component of the artificial hip joint. The cone 602 can have an opening with an inner thread, wherein an axis of the opening of the cone 602 lies on a cone axis of the cone 602. A guide rod 603 can be threaded into this opening for use in assembling the endoprosthesis and, as will be described below, in cleaning the cone 602. Anchoring stems similar to anchoring stem 601 can be used in hip, shoulder and knee joint prostheses.

The handle piece 101 of the device has a proximal end 102 and a distal end 103. When using the device to clean a cone of an implant, the handle piece 101 can be held near the proximal end 102. For a better grip, the handle piece 101 can have a plurality of axially extending grooves 111 on its outer side.

The handle piece 101 has a through opening 104 extending axially through the handle piece 101. A first section 105 of the through opening 104 is located at the distal end 103 of the handle piece 101. A second section 106 of the through opening 104 adjoins the first section 105. The first section 105 of the through opening 104 has a larger inner diameter than the second section 106. An abutment surface 107 is located at the transition between the first section 105 and the second section 106. The abutment surface 107 can be an annular surface that is perpendicular to the axial direction (vertical in FIG. 1).

The inner wall of the first section 105 of the through opening 104 can have a shape corresponding to the shape of the cone 602 of the implant 601. For example, the inner wall of the first section 105 of the through opening can have a conical shape with an opening angle corresponding to the opening angle of the cone 602, so that the textile piece 701 is pressed evenly against the cone 602 over the entire surface of the cone 602 when the handle piece 101 is placed on the cone 602. Alternatively, the first section 105 of the through opening 104 can have a cylindrical shape. Since the cone 602 of the implant 601 has a relatively small opening angle, the textile piece 701 can, due to the elasticity of the textile piece 701, also be pressed against the entire surface of the cone 602 in this case.

A wall surface of the inner wall of the first section 105 of the through opening 104 can have a corrugation. Thereby, twisting of the textile piece 701 during use of the device can be prevented or at least reduced.

Adjoined to the distal end 103 of the handle piece 101 is a distal section 114 of the handle piece 101. The distal section 114 of the handle piece 101 comprises a first region 108 that is directly adjoined to the distal end 103, a second region 109 that is further away from the distal end 103 than the first region 108, and a third region 110 that is located between the first region 108 and the second region 109. The first region 108 and the second region 109 have a larger outer diameter than the third region 110, and the outer diameter of the second region 109 can be slightly larger than the outer diameter of the first region 108.

The inner sleeve 201 has a first end 202 and a second end 203. At the first end 202 of the inner sleeve 201, the inner sleeve 201 has a protrusion 204. As shown in FIG. 1, the inner sleeve 201 is insertable into the second section 106 of the through opening 104 of the handle piece so that the second end 203 of the inner sleeve is located inside the second section 106 of the through opening 104. The protrusion 204 has an outer diameter that is larger than the inner diameter of the second section 106 of the through opening 104 in the region adjoining the first section 105 of the through opening 104, so that when the inner sleeve 201 is inserted into the second section 106 of the through opening 104, it abuts against the abutment surface 107 of the handle piece 101 and prevents the inner sleeve 201 from being slid too far into the second section 106 of the through opening.

When the device is used for cleaning a cone of an implant, a part of the textile piece 701 is located between the protrusion 204 of the inner sleeve and the abutment surface 107 of the handle piece 101, which is thereby fixed by the protrusion 204 of the inner sleeve 201 and the abutment surface 107. In this case, the inner sleeve 201 extends through an opening of the textile piece 701, as shown in FIG. 1.

The inner sleeve 201 can have slots 205, 206, 207, for example three slots. The three slots 205, 206, 207 can each be arranged offset by an angle of 120° on the circumference of the inner sleeve 201. In the region in which the slots 205, 206, 207 of the inner sleeve 201 are arranged, the wall of the inner sleeve 201 can be slightly curved outwards. When the inner sleeve 201 is inserted into the second section 106 of the through opening 104 of the handle piece 101, the outwardly curved wall is elastically deformed so that it presses on the inner wall of the second section 106 of the through opening 104. This creates friction between the inner sleeve 201 and the inner wall of the second section 106 of the through opening 104, by which the inner sleeve 201 is held against the inner wall of the second section 106 of the through opening 104. The slots 205, 206, 207 of the inner sleeve allow the wall of the inner sleeve to deform elastically more easily.

The handle piece 101 can have slots 112, 113 in the third region 110 of the distal section 114, for example, two slots. The two slots 112, 113 can be arranged offset by an angle of 180° on the circumference of the handle piece 101. When the inner sleeve 201 is inserted into the second section 106 of the through opening 104 of the handle piece 101, the slots 112, 113 of the handle piece 101 lie opposite the slots 205, 206, 207 of the inner sleeve 201 and/or the second end 203 of the inner sleeve 201. Should the inner sleeve 201 inadvertently become jammed inside the second section 106 of the through opening 104 of the handle piece 101, the inner sleeve 201 can be released using a tool inserted through one of the slots 112, 113 of the handle piece by pushing with the tool at the second end 203 of the inner sleeve 201 or at the end of one of the slots 205, 206, 207 of the inner sleeve 201.

The through opening 104 of the handle piece 101 can have an inner diameter at the proximal end 102 of the handle piece 101 that is smaller than the outer diameter of the inner sleeve 201 and smaller than the inner diameter of the second section 106 of the through opening 104 near the distal end 103 of the handle piece 101. For example, the second section 106 of the through opening 104 can taper in a region lying closer to the proximal end 102 than the region in which the inner sleeve 201 inserted into the second section 106 of the through opening 104 is located. Thus, the inner sleeve 201 cannot be inserted into the through opening 104 at the proximal end of the handle piece 102, thereby preventing improper insertion of the inner sleeve 201.

As shown in FIG. 1, the outer sleeve 301 can be slid over the distal section 114 of the handle piece 101. The first region 108 and the second region 109 of the handle piece 101 are then arranged at the ends of the outer sleeve 301. A portion of the second region 109 can then extend beyond the end of the outer sleeve 301, as shown in FIG. 1. When the device is used for cleaning the cone of an implant, a part of the textile piece 701 is located between the distal section 114 of the handle piece 101 and the inner wall 302 of the outer sleeve 301. In the first region 108 and the second region 109 of the distal section 114 of the handle piece 101, the textile piece 701 is thereby clamped between the handle piece 101 and the inner wall 302 of the outer sleeve 301, so that it is fixed.

The inner diameter of the outer sleeve 301 is slightly larger than the outer diameter of the first region 108 and the second region 109 of the distal section 114 of the handle piece 101, so that the textile piece 701 has space between the handle piece 101 and the inner wall of the outer sleeve 301. The outer diameter of the second region 109 of the distal section 114 of the handle piece 101 and the inner diameter of the outer sleeve 301 can be configured such that the second region 109 of the distal section 114 of the handle piece 101 forms a limit stop for the outer sleeve 301 when the textile piece 701 is located between the second region 109 of the distal section 114 of the handle piece 101 and the outer sleeve 301. Then, the outer sleeve 301 cannot be slid substantially further than the position shown in FIG. 1 towards the proximal end 102 of the handle piece 101. When the textile piece 701 is not properly seated, such that it is not or not sufficiently located between the second region 109 of the distal section 114 of the handle piece 101 and the inner wall 302 of the outer sleeve 301, the outer sleeve 301 can be slid beyond the position shown in FIG. 1 towards the proximal end 102 of the handle piece 101. In this way, it can be detected that the textile piece 701 is not properly inserted into the device. Thereby, the safety of use of the device is improved.

The textile piece 701 can thus be fixed between the protrusion 204 of the inner sleeve 201 and the abutment surface 107 of the handle piece 101, as well as between the regions 108, 109 of the distal section 114 of the handle piece 101 and the outer sleeve 301, so that it abuts against the inner wall of the second section 106 of the through opening 104.

The assembly tool 401 has a first end 402 and a second end 403. The first end 402 includes a first section 404, the outer diameter of which is configured to be insertable into the inner sleeve 201. Adjoined to the first section 404 is a second section 405 of the assembly tool 401, which has a larger outer diameter than the first section 404 and is insertable into the first section 105 of the through opening 104 of the handle piece. Adjoined to the second section 405 is a third section 406, which has a larger outer diameter than the second section 405 and is insertable into the outer sleeve 301. At the second end 403 of the assembly tool 401 is a standing surface 407, which allows the assembly tool 401 to be placed on a surface during assembly of the device.

The mandrel 501 has a tip 502 and a holding section 503. The holding section 503 has a smaller outer diameter than the inner diameter of the inner sleeve 201, so that it can be inserted into the inner sleeve 201 at the second end 203 of the inner sleeve 201.

The tip 502 of the mandrel 501 can have a conical shape. A portion of the mandrel 501, for example in the region of the tip 502 directly adjoining the holding section 503, has a larger outer diameter than the second section 106 of the through opening 104 of the handle piece 101, so that the mandrel 501 does not fit through the second section 106 of the through opening 104. Thereby, accidentally inserting the mandrel 501 together with the inner sleeve 201 into the second section 106 of the through opening 104 of the handle piece 101 is prevented. Thereby, the safety of use of the device is improved.

For assembling the device, first the standing surface 407 of the assembly tool 401 is placed on a flat surface and the outer sleeve 301 is slid onto the assembly tool 401 so that the third section 406 of the assembly tool 401 is located in the outer sleeve 301. The inner sleeve 201 is then placed on the first section 404 of the assembly tool 401, and the holding section 503 of the mandrel 501 is inserted into the second end 203 of the inner sleeve 201. The textile piece 701 is then placed on the tip 502 of the mandrel 501 so that the latter is located approximately in the center of the textile piece 701, and the textile piece 701 is slid downwards until it rests on the protrusion 204 of the inner sleeve 201. In doing so, it is pierced by the tip 502 of the mandrel 501 so that the textile piece 701 has an opening, the edge of which rests on the protrusion 204 of the inner sleeve 201. The mandrel 501 is then removed and the handle piece 101 is slid over the inner sleeve 201 until the protrusion 204 of the inner sleeve, on which the textile piece 701 abuts, rests against the abutment surface 107 of the handle piece 101. The outer sleeve 301 is then slid upwards until it is in the position shown in FIG. 1.

The device can then be used to clean the cone 602 of the implant 601. For this purpose, it is placed on the cone 602 of the implant 601 with the distal end 103 of the handle piece 101 leading. The guide rod 603, which is connected to the implant 601 as shown in FIG. 7, is thereby inserted into the through opening 104 of the handle piece 101 as well as into the inner sleeve 201. Then, the device is rotated so that the cone 602 is cleared of impurities from the part of the textile piece 701 abutting the inner wall of the first section 105 of the through opening 104 of the handle piece 101. The device can then be removed from the cone 602 of the implant and the guide rod 603, and a module of the endoprosthesis with a cone-shaped recess matching the cone 602 can be placed on the cleaned cone 602 to form a cone connection.

What is claimed is:

1. A device for cleaning a cone of an implant, comprising:
a handle piece with a proximal end, a distal end, and a through opening extending axially through the handle piece, wherein the through opening has, at the distal end of the handle piece, a first section which has a larger inner diameter than a second section of the through opening adjoining the first section;
an inner sleeve insertable into the second section of the through opening of the handle piece and having, at a first end, a protrusion for abutment against an abutment surface of the handle piece located at a transition between the first and the second section of the through opening of the handle piece; and
an outer sleeve slidable over a distal section of the handle piece at the distal end of the handle piece;
wherein the handle piece and the inner sleeve are adapted to fix a first part of a textile piece between the protrusion of the inner sleeve and the abutment surface of the handle piece upon insertion of the inner sleeve into the second section of the through opening of the handle piece; and
wherein the handle piece and the outer sleeve are adapted to fix a second part of the textile piece between the distal section of the handle piece and the outer sleeve when the outer sleeve is slid over the distal section of the handle piece.

2. The device according to claim 1, additionally comprising:
an assembly tool comprising, at a first end, a first section insertable into the inner sleeve, and a second section adjoining the first section, which has a larger outer diameter than the first section and is insertable into the first section of the through opening of the handle piece, and a third section adjoining the second section, which has a larger outer diameter than the second section and is insertable into the outer sleeve.

3. The device according to claim 2, wherein:
the assembly tool has a standing surface at a second end for placing the assembly tool on a surface.

4. The device according to claim 1, additionally comprising:
a mandrel with a tip configured to pierce the textile piece, wherein the mandrel has at an end facing away from the tip a holding section which is insertable into the inner sleeve at the second end of the inner sleeve facing away from its first end.

5. The device according to claim 4, wherein:
a part of the mandrel has a larger outer diameter than the second section of the through opening of the handle piece, so that the mandrel does not fit through the second section of the through opening.

6. The device according to claim 1, wherein:
the inner sleeve has one or more slots.

7. The device according to claim 6, wherein:
the inner sleeve in the region where the slots are arranged has an outwardly curved wall having an elasticity configured to hold the inner sleeve inserted into the second section of the through opening of the handle piece against the inner wall of the second section of the through opening of the handle piece.

8. The device according to claim 1, wherein:
the distal section of the handle piece has a first region and a second region, each having a larger outer diameter than a third region arranged between the first and the second region, wherein the first and second regions of the handle piece are arranged at the ends of the outer sleeve when the outer sleeve is slid over the distal section of the handle piece.

9. The device according to claim 8, wherein:
the distal section of the handle piece in the third region has one or more slots arranged opposite the second end of the inner sleeve and/or one or more slots of the inner sleeve when the inner sleeve is inserted into the second section of the through opening of the handle piece.

10. The device according to claim 8, wherein:
the second region of the distal section of the handle piece is further away from the distal end of the handle piece than the first region of the distal section of the handle piece, and the outer diameter of the second region of the distal section of the handle piece and an inner diameter of the outer sleeve are configured such that the second region of the distal section of the handle piece forms a limit stop for the outer sleeve, when the textile piece is located between the second region of the distal section of the handle piece and the outer sleeve and the outer sleeve is slidable beyond the second region of the distal section of the handle piece when the textile piece is not located between the second region of the distal section of the handle piece and the outer sleeve.

11. The device according to claim 1, wherein:
the handle piece at the proximal end has a plurality of axially extending grooves on the outer side of the handle piece.

12. The device according to claim 1, wherein:
the through opening of the handle piece at the proximal end of the handle piece has an inner diameter that is smaller than an outer diameter of the inner sleeve and smaller than the inner diameter of the second section of the through opening at the distal end of the handle piece.

13. The device according to claim 1, wherein:
the first section of the through opening has a corrugation on its wall surface.

14. The device according to claim 1, wherein:
the textile piece is a gauze compress.

15. A device for cleaning a cone of an implant, comprising:
a handle piece having a proximal end, a distal end and a through opening extending axially through the handle piece, wherein the through opening has, at the distal end of the handle piece, a first section which has a larger inner diameter than a second section of the through opening adjoining the first section, wherein an abutment surface is located at a transition between the first and the second section of the through opening of the handle piece;
an inner sleeve which is inserted into the second section of the through opening of the handle piece and which has a protrusion at a first end;
an outer sleeve which encloses a distal section of the handle piece at the distal end of the handle piece; and
a textile piece having an opening through which the inner sleeve is passed, wherein a first part of the textile piece is fixed between the protrusion of the inner sleeve and the abutment surface of the handle piece and a second part of the textile piece is fixed between the distal section of the handle piece and an inner wall of the outer sleeve.

16. A surgical system comprising:
a device for cleaning a cone of an implant, comprising:
a handle piece with a proximal end, a distal end and a through opening extending axially through the handle piece, wherein the through opening has, at the distal end of the handle piece, a first section which has a larger inner diameter than a second section of the through opening adjoining the first section;
an inner sleeve insertable into the second section of the through opening of the handle piece and having, at a first end, a protrusion for abutment against an abutment surface of the handle piece located at the transition between the first and the second section of the through opening of the handle piece; and
an outer sleeve slidable over a distal section of the handle piece at the distal end of the handle piece;
wherein the handle piece and the inner sleeve are adapted to fix a first part of a textile piece between the protrusion of the inner sleeve and the abutment surface of the handle piece upon insertion of the inner sleeve into the second section of the through opening of the handle piece; and
wherein the handle piece and the outer sleeve are adapted to fix a second part of the textile piece between the distal section of the handle piece and the outer sleeve when the outer sleeve is slid over the distal section of the handle piece; and
an implant having a cone, wherein the cone of the implant and the first section of the through opening of the handle piece of the device for cleaning the cone of an implant have corresponding shapes.

17. The surgical system according to claim 16, wherein the implant is an anchoring stem of an endoprosthesis.

18. The surgical system according to claim 16, wherein:
the cone of the implant has an opening with an inner thread, wherein an axis of the opening of the cone of the implant lies on a cone axis of the cone, and
the system additionally comprises a guide rod, which can be screwed into the opening of the cone of the implant, and wherein the guide rod can be guided through the through opening of the handle piece and the inner sleeve of the device for cleaning a cone of an endoprosthesis that is inserted into the second section of the through opening of the handle piece.

19. A method, comprising:
providing a device for cleaning a cone of an implant, comprising:
a handle piece with a proximal end, a distal end and a through opening extending axially through the handle piece, wherein the through opening has, at the distal end of the handle piece, a first section which has a larger inner diameter than a second section of the through opening adjoining the first section;
an inner sleeve insertable into the second section of the through opening of the handle piece and having at a first end a protrusion for abutment against an abutment surface of the handle piece located at a transition between the first and the second section of the through opening of the handle piece; and
an outer sleeve slidable over a distal section of the handle piece at the distal end of the handle piece;
inserting the inner sleeve into an opening in a textile piece, wherein an edge of the opening of the textile piece rests on the protrusion of the inner sleeve;
inserting the inner sleeve into the second section of the through opening of the handle piece, wherein a first part of the textile piece is fixed between the protrusion of the inner sleeve and the abutment surface of the handle piece; and
sliding the outer sleeve over the distal section of the handle piece, wherein a second part of the textile piece is fixed between the distal section of the handle piece and the outer sleeve.
